(19) United States
(12) Patent Application Publication
(10) Pub. No.: US 2021/0277359 A1
(43) Pub. Date: Sep. 9, 2021

(54) CELL AND TISSUE MODELS COMPRISING FIBROBLASTS OF THE DERMO-HYPODERMIC JUNCTION AND APPLICATIONS THEREOF

(71) Applicant: L'OREAL, PARIS (FR)

(72) Inventor: Valérie HAYDONT, Aulnay-Sous-Bois (FR)

(21) Appl. No.: 16/973,916

(22) PCT Filed: Jul. 1, 2019
(86) PCT No.: PCT/EP2019/067638

§ 371 Date: Dec. 10, 2020

(30) Foreign Application Priority Data
Jun. 29, 2018 (FR) 1855987

(51) Int. Cl. C12N 5/077; C12Q 1/6881

(52) U.S. Cl. CPC C12N 5/0656; C12N 2535/00; C12N 2533/54; C12Q 1/6881

(57) ABSTRACT

The present invention relates to an in vitroprocess for preparing cell and tissue models comprising at least one step of culturing fibroblasts of the dermo-hypodermic junction; the models obtained and also the implementation thereof in processes for screening active agents that promote the differentiation of fibroblasts of the dermo-hypodermic junction into adipocyte, osteoblast and/or chondroblast cells.

The present invention relates to novel cellular and tissue models comprising fibroblasts of the dermo-hypodermic junction, and also processes for the preparation thereof.

Furthermore, the invention also relates to the use of these models as tools for screening active agents that promote the differentiation of fibroblasts into adipocyte, osteoblast and/or chondroblast cells.

The skin is constituted of three linked compartments, namely the epidermis, dermis and hypodermis.

The epidermis is predominantly constituted of three cell types, namely keratinocytes, themselves predominant among the cells of the epidermis, melanocytes and Langerhans cells. These cells constitute a keratinized epithelium that differentiates into superimposed layers surmounted by a layer of dead cells forming the stratum corneum.

The hypodermis is constituted of connective trabeculae enclosing fat lobules formed by the accumulation of adipocytes. The connective trabeculae stabilize the cohesion of the tissue while facilitating the passage of vessels. The primary functions of the hypodermis are ensuring thermal regulation of the body but also storage of energy molecules and toxins.

Epidermis and hypodermis surround the dermis. The latter provides a solid support to the epidermis. It is also its nourishing element. It is mainly constituted of fibroblasts and an extracellular matrix.

Fibroblasts, often nicknamed “support cells”, are present in many connective tissues, and in particular in the skin, tendons, cartilage, bone tissue, etc.

At present, only two subpopulations of fibroblasts in the skin have been identified in the literature: papillary fibroblasts and reticular fibroblasts.

Their potential as a screening tool has been highlighted by in vitro studies showing their ability to differentiate into other cell types forming connective tissues such as fat cells (adipocytes), cartilage cells (chondroblasts) and bone cells (osteoblasts).

Thus, models that make it possible to screen active agents that promote the differentiation of fibroblasts into adipocytes, chondroblasts, and/or osteoblasts have already been described in the literature. Mention may especially be made of Brun C et al. “Intrinsically aged dermal fibroblasts fail to differentiate into adipogenic lineage” Exp Dermatol. 2016 November 25(11):906-909; Chen F G et al. “Clonal analysis of nestin(−) vimentin(+) multipotent fibroblasts isolated from human dermis”, J Cell Sci. 2007 Aug. 15 120(Pt 16):2875-83; Jeney F et al. “Cytochemical studies on the fibroblast-preadipocyte relationships in cultured fibroblast cell lines” Acta Histochem. 2000 November; 102(4):381-9.

Aside from the use for studying the differentiation capacity of fibroblasts, such models also prove beneficial in the study of skin aging. Indeed, it is known that skin aging is correlated with a loss of the capacity of fibroblasts to differentiate into adipocytes (Cécilia Brun et al. “Altération de la différenciation adipogénique des fibroblastes dermiques avec l'âege” [“Alteration in the adipogenic differentiation of dermal fibroblasts with age”], annales de Dermatologie et Vénérologie, vol 143, December 2016).

However, these models have proven to be not sufficiently effective, especially not sufficiently sensitive, in that they generate false negatives for the active agents, as shown in the comparisons below.

There therefore remains a need to provide more effective and/or more sensitive cell or tissue models which make it possible to avoid obtaining false negatives during their use in processes for screening active agents that promote the differentiation of fibroblasts into adipocyte, osteoblast and/or chondroblast cells.

The Applicant has demonstrated that the use of a particular subpopulation of dermal fibroblasts, the fibroblasts of the dermo-hypodermic junction, makes it possible to obtain cell or tissue models that promote the differentiation of these cells into adipocytes, chondroblasts and/or osteoblasts that are much more sensitive than those disclosed in the prior art, and in particular makes it possible to avoid the false negatives generated by an excessive proportion of non-responder cells of papillary and/or reticular fibroblast type.

Thus, a first subject of the present invention is an in vitro process for preparing a cell model comprising at least one step of culturing fibroblasts of the dermo-hypodermic junction.

The present invention also relates to an in vitro process for preparing a tissue model comprising at least one step of culturing fibroblasts of the dermo-hypodermic junction on collagen sponge.

According to another subject thereof, the present invention relates to cell and tissue models obtained by said processes.

The present invention also relates to a method of screening for active agents that promote the differentiation of fibroblasts of the dermo-hypodermic junction into adipocyte, osteoblast and/or chondroblast cells using said cell and tissue models.

### DETAILED DESCRIPTION OF THE INVENTION

### Fibroblasts of the Dermo-Hypodermic Junction

“Fibroblast of the dermo-hypodermic junction” or “DHJF fibroblast” is intended here to mean a fibroblast of the region located at the connective trabeculae projecting from the dermis into the hypodermis. DHJF fibroblasts in culture typically have a highly heterogeneous morphology. Thus, highly varied forms can be observed in the cell layer ranging from very small tricuspid cells, to very large multipolar cells having a highly pronounced intracellular trabecular network (visible in light microscopy).

Aside from their morphological characteristics and their location as mentioned above, the fibroblasts of the dermo-hypodermic junction according to the invention may be identified by measuring the level of expression of at least one gene selected from the group consisting of the genes UCP2, ACAN, FGF9 and COL11A1, and the level of an expression product of the gene KLF9.

In particular, the method for identifying fibroblasts as being fibroblasts of the dermo-hypodermic junction comprises:
a) providing a biological sample comprising at least one dermal fibroblast,
b) measuring, in the biological sample provided in step a), the level of an expression product of at least one gene selected from the group consisting of the genes UCP2, ACAN, FGF9 and COL11A1, and the level of an expression product of the gene KLF9,
c) identifying the dermal fibroblast of step a) as a fibroblast of the dermo-hypodermic junction when:
1) (i) the level of the expression product of the gene UCP2 is decreased relative to a control level,
   (ii) the level of the expression product of the gene ACAN is increased relative to a control level,
   (iii) the level of the expression product of the gene FGF9 is increased relative to a control level, and/or
   (iv) the level of the expression product of the gene COL11A1 is increased relative to a control level,
and
2) the level of the expression product of the gene KLF9 is increased relative to a control level,
   the control levels of 1(i), 1(ii), 1(iii) and 1(iv) preferably being respectively the level of the expression product of the genes UCP2, ACAN, FGF9 and COL11A1 in a dermal fibroblast known to be a papillary fibroblast and the control level of 2) preferably being the level of the expression product of the gene KLF9 in a dermal fibroblast known to be a reticular fibroblast.

Advantageously, the biological sample provided in step a) is an in vitro culture of dermal fibroblasts or a mixture of dermal fibroblasts, a sample originating from a skin biopsy, or a sample originating from a dermis or skin equivalent obtained in vitro.

Preferably, the biological sample of dermal fibroblasts is isolated from non-defatted human skin, at the connective trabeculae present at the dermo-hypodermic junction. The latter are taken using tweezers and scissors.

In particular, the sample originates from a human skin biopsy performed on young subjects, such as subjects between 15 and 40 years, preferably between 17 and 31 years.

“Expression product of the gene X” is intended here to mean the mRNA encoded by said gene X or the protein encoded by said gene X. The level of the expression product of the gene X may therefore be measured by quantifying the corresponding mRNA or protein. In a particular embodiment, said expression product of the gene X is the mRNA encoded by said gene X.

Preferably, the level of the expression product corresponds to the concentration or the amount of the expression product.

The level of the expression product of the gene X in the sample tested is said to be increased when the ratio [level of expression of the gene X in the sample tested/control level of the gene X] is greater than or equal to 2.

The level of the expression product of the gene X in the sample tested is said to be decreased when the ratio [control level of the gene X/level of expression of the gene X in the sample tested] is greater than or equal to 2.

When the ratio [control level of the gene X/level of expression of the gene X in the sample tested] is greater than or equal to 2, the sign (−) is placed in front of the value obtained.

This ratio is more commonly known as “fold change”.

“Control level” is intended here to mean a reference value, preferably corresponding to the level of said expression product of the gene in a dermal fibroblast known to be a papillary or reticular dermal fibroblast originating from the same donor.

“Dermal fibroblast known to be a papillary or reticular dermal fibroblast” is intended here to mean a dermal fibroblast of which the type (papillary or reticular) has previously been determined in light of its morphology, its origin or biomarkers detected.

In particular, the dermal fibroblasts known to be papillary or reticular dermal fibroblasts are taken as follows:
a) the papillary fibroblasts (Fp), reticular fibroblasts (Fr) were isolated from non-defatted human skin;
b) the isolation of the Fr is carried out on the part of tissue thus depleted of its dermo-hypodermic junction, the tissue is then dermatomed to 700 μm. Only the lower part of the tissue is retained; and or
c) the isolation of the Fp is carried out on tissue dermatomed to 300 μm and de-epidermized after dispase action (Roche—2.4 unit/ml) for 16 h at 4° C.

The level of the expression product of a gene X may be measured in step (b) of the abovementioned identification method by any technique well-known to those skilled in the art. In particular, when the expression product is a protein, the level of the expression product may be measured by means of immunological assays such as ELISA assays, immunofluorescent assays (IFA), radioimmunoassays (RIA), competitive binding assays or Western blots. When the expression product is an mRNA, the level of the expression product can be measured by RT-PCR, qRT-PCR, ddPCR (Droplet Digital PCR), by sequencing, for example by sequencing of NGS type Next Generation Sequencing) or by ddSEQ™ single-cell isolator sequencing.

“Dermal fibroblast” is intended here to mean any fibroblast originating from the dermis.

“Papillary fibroblast” is intended here to mean a fibroblast of the papillary dermis, the papillary dermis being characterized by a relatively thin extracellular matrix and a high cell density and is located, at the tissue level, between the epidermis and the superficial plexus, also referred to as rete subpapillae. Papillary fibroblasts in culture typically have a thin and fusiform morphology.

“Reticular fibroblast” is intended here to mean a fibroblast of the reticular dermis, the reticular dermis being characterized by a very dense network of matrix fibers and a low cell density. At the tissue level, it extends from the superficial plexus to the deep plexus, also referred to as rete cutaneum. Reticular fibroblasts in culture typically have an extended and more square appearance.

“Gene UCP2” is intended here to mean the gene encoding the “mitochondrial uncoupling protein 2”. The gene UCP2 is also referred to as the gene SLC25A8 and the protein UCP2 is also referred to as UCPH or “solute carrier family 25 member 8”. It belongs to the family of mitochondrial anionic carrier proteins (MACP) and proteins for controlling mitochondria-derived reactive oxygen species. It is typically described in Pecqueur et al. (1999) Biochemical and Biophysical Research Communications 255: 40-46. The human UCP2 protein sequence is typically referenced under the UniProt number P55851.

“Gene ACAN” is intended here to mean the gene encoding the “aggrecan core protein”. The gene ACAN is also referred to as gene AGC1, CSPG1 and MSK16 and the ACAN protein is also referred to as “aggrecan” or “cartilage-specific proteoglycan core protein” or CSPCP or “chondroitin sulfate proteoglycan core protein 1” or “chondroitin sulfate proteoglycan 1”. It is part of the extracellular matrix in cartilaginous tissue. It is a proteoglycan. It is typically described in Doege et al. (1991) J. Biol. Chem. 15: 894-902. The human ACAN protein sequence is typically referenced under the UniProt number P16112.

“Gene FGF9” is intended here to mean the gene encoding fibroblast growth factor 9. Protein FGF9 is also referred to as “Glia-activating factor” or GAF or “heparin-binding growth factor 9” or HBGF-9. It has a growth stimulating effect on glial cells in culture. It is typically described in Miyamoto et al. (1993) Molecular and Cellular Biology 13: 4251-4259. The human FGF9 protein sequence is typically referenced under the UniProt number P31371.

“Gene COL11A1” is intended here to mean the gene encoding the α1 (XI) collagen chain. The gene COL11A1 is also referred to as gene COLL6. This chain is one of the two alpha chains of type XI collagen, a minor fibrillar collagen. It is typically described in Yoshioka et al. (1990) J. Biol. Chem. 15: 6423-6426. The human COL11A1 protein sequence is typically referenced under the UniProt number P12107.

“Gene KLF9” is intended here to mean the gene encoding the “Krueppel-like factor 9”. The gene KLF9 is also referred to as gene BTEB or gene BTEB1 and the protein KLF9 is also referred to as BTEB1 transcription factor or “GC-box-binding protein 1” or “basic transcription element-binding” protein 1″ or “BTE-binding protein 1”. It is part of the zinc finger transcription factor family of type Sp1 C2H2. It is typically described in Spörl et al. (2012) Proc. Natl. Acad. Science USA 109: 10903-10908. The human KLF9 protein sequence is typically referenced under the UniProt number Q13886.

In the context of the invention, the UniProt references cited above are those that were available as of Jun. 19, 2018.

### Cell Model

Process for Preparing the Cell Model

A subject of the present invention is an in vitro process for preparing a cell model comprising at least one step of culturing fibroblasts of the dermo-hypodermic junction.

Preferably, said fibroblasts of the dermo-hypodermic junction are identified in accordance with the identification method as defined according to the section “Fibroblasts of the dermo-hypodermic junction”.

Said fibroblasts are in particular seeded in a number of between 700 and 5000 cells per cm², preferably between 1000 and 4000 cells per cm².

In addition, the culture support used is a 2D culture support well known to those skilled in the art, for example a plastic petri dish treated for cell culture or multi-well culture plates such as 24- or 96-well microplates.

Advantageously, said fibroblasts are cultured between 24 h and 72 h after reaching at least 80% confluence, preferably for 48 h after reaching at least 80% confluence, even better still for 48 h after reaching confluence.

“Confluence” is intended to mean a cell layer having no interstices between each adherent cell cultured in a monolayer on a suitable support and may for example be observed with the naked eye or microscope.

Said fibroblasts of the dermo-hypodermic junction are preferably cultured in a culture medium allowing their amplification, in particular a medium selected from MEM, DMEM, DMEM/F12, and/or FGM further comprising at least 5% fetal calf serum (FCS), glutamine, sodium pyruvate, non-essential amino acids and optionally antibiotics and/or antimycotics.

In a particular embodiment, said fibroblasts of the dermo-hypodermic junction are cultured in an MEM culture medium further comprising 10% fetal calf serum (FCS), glutamine, sodium pyruvate, non-essential amino acids and optionally antibiotics and/or antimycotics.

“Amplification” is intended here to mean the proliferation or multiplication of the cells.

In a first embodiment, the in vitro process for preparing a cell model according to the invention also comprises a step of centrifugation of the fibroblasts obtained at the end of said culture step.

In a second embodiment, the in vitro process for preparing a cell model according to the invention does not comprise a step of centrifugation of the fibroblasts obtained at the end of said culture step.

In a particular embodiment, the process for preparing the cell model according to the invention does not comprise carrying out a step of culturing fibroblasts other than those of the dermo-hypodermic junction, in particular it does not comprise a step of culturing papillary and/or reticular fibroblasts.

In another embodiment, the step of culturing fibroblasts of the dermo-hypodermic junction in the preparation process according to the invention is carried out from a biological sample comprising an amount of fibroblasts of the dermo-hypodermic junction of at least 60%, preferably at least 80%, relative to the total amount of cells present in said biological sample.

Even more preferably, the step of culturing fibroblasts of the dermo-hypodermic junction in the preparation process according to the invention is carried out from a biological sample comprising an amount of fibroblasts of the dermo-hypodermic junction of between 60% and 95%, even better still between 80% and 95%, relative to the total amount of cells present in said biological sample.

The biological sample may be an in vitro culture of dermal fibroblasts or a mixture of dermal fibroblasts, a sample originating from a skin biopsy, or a sample originating from a dermis or skin equivalent obtained in vitro.

Preferably, the biological sample of dermal fibroblasts is isolated from non-defatted human skin, at the connective trabeculae present at the dermo-hypodermic junction. The latter are taken using tweezers and scissors.

Cell Model

The present invention also relates to a cell model obtainable according to the process mentioned above in the subsection “Process for preparing the cell model”.

Preferably, the cell model according to the invention comprises fibroblasts of the dermo-hypodermic junction, for which:
1) (i) the level of the expression product of the gene UCP2 is decreased relative to a control level,
   (ii) the level of the expression product of the gene ACAN is increased relative to a control level,
   (iii) the level of the expression product of the gene FGF9 is increased relative to a control level, and/or
   (iv) the level of the expression product of the gene COL11A1 is increased relative to a control level,
and
2) the level of the expression product of the gene KLF9 is increased relative to a control level.
the control levels of 1(i), 1(ii), 1(iii) and 1(iv) preferably being respectively the level of the expression product of the genes UCP2, ACAN, FGF9 and COL11A1 in a dermal fibroblast known to be a papillary fibroblast and the control level of 2) preferably being the level of the expression product of the gene KLF9 in a dermal fibroblast known to be a reticular fibroblast.

The definitions of the terms “control level”, “dermal fibroblast known to be a papillary or reticular dermal fibroblast”, “expression product of the gene X”, “dermal fibroblast”, “papillary fibroblast”, “reticular fibroblast”, “gene UCP2”, “gene ACAN”, “gene FGF9”, “gene COL11A1”, and “gene KLF9” mentioned above in the section “Fibroblasts of the dermo-hypodermic junction” will be used again.

When the process according to the invention also comprises a step of centrifugation of the fibroblasts obtained at the end of said culture step, then the cell model is in the form of a spheroid.

“Spheroid” is intended to mean a cluster of cells of irregular and unorganized shape.

In a particular embodiment, the cell model according to the invention is constituted of fibroblasts of the dermo-hypodermic junction, for which:
1) (i) the level of the expression product of the gene UCP2 is decreased relative to a control level,
   (ii) the level of the expression product of the gene ACAN is increased relative to a control level,
   (iii) the level of the expression product of the gene FGF9 is increased relative to a control level, and/or
   (iv) the level of the expression product of the gene COL11A1 is increased relative to a control level,
and
2) the level of the expression product of the gene KLF9 is increased relative to a control level.
the control levels of 1(i), 1(ii), 1(iii) and 1(iv) preferably being respectively the level of the expression product of the genes UCP2, ACAN, FGF9 and COL11A1 in a dermal fibroblast known to be a papillary fibroblast and the control level of 2) preferably being the level of the expression product of the gene KLF9 in a dermal fibroblast known to be a reticular fibroblast.

In another particular embodiment, the fibroblasts of the dermo-hypodermic junction are present in the cell model according to the invention in an amount of at least 60%, preferably at least 80%, relative to the total amount of cells present in said cell model.

Even more preferably, the fibroblasts of the dermo-hypodermic junction are present in the cell model according to the invention in an amount of between 60% and 95%, even better still between 80% and 95%, relative to the total amount of cells present in said cell model.

### Tissue Model

### Process for Preparing the Tissue Model

The present invention also relates to an in vitro process for preparing a tissue model comprising at least one step of culturing fibroblasts of the dermo-hypodermic junction on collagen sponge.

The term “collagen sponge” is also well known to those skilled in the art. It refers to biopolymers based on collagen.

According to the invention, the collagen may be any type of collagen of any origin, preferentially of animal origin, in particular of bovine origin. In this regard, reference will be made to the different types of collagen mentioned in the reviews of Van der Rest and Garonne, 1990, Biochem., Vol. 72, 473-484 or 1991, Faseb Journal, Vol. 5, 2814-2823.

Thus, according to the invention, the collagen is preferably selected from type I, III or V fibrillar collagens; even better still, the collagen used in the context of the present invention is type I collagen.

Mention may be made, as example of collagen sponge, of the Mimedisk® sponges sold by BASF Beauty Care, or else Symatese Biomateriaux.

Said fibroblasts are preferably seeded in a number of between 125 000 and 500 000 cells, preferably between 200 000 and 300 000 cells.

Preferably, said fibroblasts are cultured for 10 to 20 days, particularly preferably for 14 days.

Advantageously, the tissue model according to the invention is not in the form of a collagen lattice. The term “collagen lattice” is well known in the state of the art (Bell et al., 1979, Proc Natl Acad Sci USA, Vol. 76, No. 3, pp. 1274-1278) and it refers to a dermis equivalent model known and used for decades.

Said fibroblasts of the dermo-hypodermic junction are preferably cultured in a culture medium allowing their amplification, in particular a medium selected from MEM, DMEM, DMEM/F12, and/or FGM further comprising at least 5% fetal calf serum (FCS), glutamine, sodium pyruvate, non-essential amino acids and optionally antibiotics and/or antimycotics.

In a particular embodiment, said fibroblasts of the dermo-hypodermic junction are cultured in an MEM culture medium further comprising 10% fetal calf serum (FCS), glutamine, sodium pyruvate, non-essential amino acids and optionally antibiotics and/or antimycotics.

“Amplification” is intended here to mean the proliferation or multiplication of the cells.

In a particular embodiment, the process for preparing the tissue model according to the invention does not comprise carrying out a step of culturing fibroblasts other than those of the dermo-hypodermic junction, in particular it does not comprise a step of culturing papillary and/or reticular fibroblasts.

In another embodiment, the step of culturing fibroblasts of the dermo-hypodermic junction in the preparation process according to the invention is carried out from a biological sample comprising an amount of fibroblasts of the dermo-hypodermic junction of at least 60%, preferably at least 80%, relative to the total amount of cells present in said biological sample.

Even more preferably, the step of culturing fibroblasts of the dermo-hypodermic junction in the preparation process according to the invention is carried out from a biological sample comprising an amount of fibroblasts of the dermo-hypodermic junction of between 60% and 95%, even better still between 80% and 95%, relative to the total amount of cells present in said biological sample.

The biological sample may be an in vitro culture of dermal fibroblasts or a mixture of dermal fibroblasts, a sample originating from a skin biopsy, or a sample originating from a dermis or skin equivalent obtained in vitro.

Preferably, the biological sample of dermal fibroblasts is isolated from non-defatted human skin, at the connective trabeculae present at the dermo-hypodermic junction. The latter are taken using tweezers and scissors.

### Tissue Model

The present invention also relates to a tissue model obtainable according to the process mentioned above in the subsection “Process for preparing the tissue model”.

Preferably, the tissue model according to the invention comprises fibroblasts of the dermo-hypodermic junction, for which:
1) (i) the level of the expression product of the gene UCP2 is decreased relative to a control level,
   (ii) the level of the expression product of the gene ACAN is increased relative to a control level,
   (iii) the level of the expression product of the gene FGF9 is increased relative to a control level, and/or
   (iv) the level of the expression product of the gene COL11A1 is increased relative to a control level,
and
2) the level of the expression product of the gene KLF9 is increased relative to a control level.
the control levels of 1(i), 1(ii), 1(iii) and 1(iv) preferably being respectively the level of the expression product of the genes UCP2, ACAN, FGF9 and COL11A1 in a dermal fibroblast known to be a papillary fibroblast and the control level of 2) preferably being the level of the expression product of the gene KLF9 in a dermal fibroblast known to be a reticular fibroblast.

The definitions of the terms “control level”, “dermal fibroblast known to be a papillary or reticular dermal fibroblast”, “expression product of the gene X”, “dermal fibroblast”, “papillary fibroblast”, “reticular fibroblast”, “gene UCP2”, “gene ACAN”, “gene FGF9”, “gene COL11A1”, and “gene KLF9” mentioned above in the section “Fibroblasts of the dermo-hypodermic junction” will be used again.

In a particular embodiment, the tissue model according to the invention is constituted of fibroblasts of the dermo-hypodermic junction, for which:
1) (i) the level of the expression product of the gene UCP2 is decreased relative to a control level,
   (ii) the level of the expression product of the gene ACAN is increased relative to a control level,
   (iii) the level of the expression product of the gene FGF9 is increased relative to a control level, and/or
   (iv) the level of the expression product of the gene COL11A1 is increased relative to a control level,
and
2) the level of the expression product of the gene KLF9 is increased relative to a control level.
the control levels of 1(i), 1(ii), 1(iii) and 1(iv) preferably being respectively the level of the expression product of the genes UCP2, ACAN, FGF9 and COL11A1 in a dermal fibroblast known to be a papillary fibroblast and the control level of 2) preferably being the level of the expression product of the gene KLF9 in a dermal fibroblast known to be a reticular fibroblast.

In another particular embodiment, the fibroblasts of the dermo-hypodermic junction are present in the tissue model according to the invention in an amount of at least 60%, preferably at least 80%, relative to the total amount of cells present in said tissue model. Even more preferably, the fibroblasts of the dermo-hypodermic junction are present in the tissue model according to the invention in an amount of between 60% and 95%, even better still between 80% and 95%, relative to the total amount of cells present in said tissue model.

### Use and Screening Process

The present invention also relates to the use of in vitro cell or tissue models as defined in the sections “Cell model” and “Tissue model” as tool for screening for active agents that promote the differentiation of fibroblasts of the dermo-hypodermic junction into adipocyte, osteoblast and/or chondroblast cells.

“Adipocyte” is intended to mean cells characterized by an accumulation of lipid droplets within their cell bodies, these droplets being able to fuse to constitute a single lipid vacuole which will occupy the whole volume of the cell. Their presence is often confirmed by the use of lipid stain such as red oil (Oil Red O) or Sudans (Sudan black).

“Osteoblast” is intended to means cells, the morphology of which does not differ from that of fibroblasts in two-dimensional culture. Their presence is generally confirmed by the use of stain revealing the synthesis of extracellular matrix typical of that of bone mineralization. For this purpose, stains such as alizarin red are used. The latter has a high affinity for calcium deposits. It is also possible to assay the alkaline phosphatase activity (an enzyme strongly present within these cells).

“Chondroblast” is intended to means cells, the morphology of which does not differ from that of fibroblasts in two-dimensional culture. The presence of chondroblasts is generally demonstrated by the use of stain having a high affinity for the extracellular matrix that they secrete. Among the commonly used stains, mention may be made of Toluidine blue and Safranin O. These have a high affinity for the acidic proteoglycans highly present in cartilage. After fixing, the Safranin O stains orange or even red, whereas Toluidine blue will exhibit violet staining. Immunohistochemical labeling directed against proteins strongly present in this extracellular matrix may also be carried out. Mention may thus be made of type II collagen, type XI collagen or else aggrecan.

The present invention also relates to a process for screening active agents that promote the differentiation of fibroblasts of the dermo-hypodermic junction into adipocyte, osteoblast and/or chondroblast cells, comprising the following steps:
i. providing a cell or tissue model as defined according to the sections “Cell model” and “Tissue model”,
ii. bringing said model into contact with at least one agent to be screened,
iii. carrying out a qualitative and/or quantitative measurement of the expression of at least one marker of the adipocyte, osteoblast and/or chondrocyte cells or of their biological activity (activities), then
iv. comparing the measurement carried out in step iii) with that obtained from a control.

Advantageously, the control is a cell or tissue model cultured under the same conditions as that implemented in step i) but which has not received the active agent to be screened.

According to a first embodiment, said model in step i) is a cell model obtainable according to the preparation process comprising at least one step of culturing fibroblasts of the dermo-hypodermic junction and makes it possible to carry out, in step iii), a qualitative and/or quantitative measurement of the expression of at least one marker of the adipocyte and/or osteoblast cells or of their biological activity (activities).

According to a second embodiment, said model in step i) is a cell model obtainable according to the preparation process comprising at least one step of culturing fibroblasts of the dermo-hypodermic junction followed by a step of centrifugation and makes it possible to carry out, in step iii), a qualitative and/or quantitative measurement of the expression of at least one marker of the chondroblast cells or of their biological activity (activities).

According to a third embodiment, said model in step i) is a tissue model obtainable according to the preparation process comprising at least one step of culturing fibroblasts of the dermo-hypodermic junction on collagen sponge and makes it possible to carry out, in step iii), a qualitative and/or quantitative measurement of the expression of at least one marker of the adipocyte and/or osteoblast cells or of their biological activity (activities).

“Qualitative and/or quantitative measurement of the expression of at least one marker of the adipocyte, osteoblast and/or chondroblast cells” is intended to mean equally the morphological characteristics visible to the naked eye or under the microscope and also the presence and/or expression of genes and/or proteins specific to a given cell type (adipocyte, osteoblast or chondroblast) as mentioned above in the section “Use and screening process” and also in the following publications: Pittenger et al. Science 1999, Peiffer et al. Leukemia 2007, Choudhery et al. J Transl Med 2014.

“Biological activity of the adipocyte, osteoblast and/or chondroblast cells” is intended to mean an enzymatic activity and/or a metabolite production activity specific to a given cell type (adipocyte, osteoblast or chondroblast) as mentioned above in the section “Use and screening process” and also in the following publications: Pittenger et al. Science 1999, Peiffer et al. Leukemia 2007, Choudhery et al. J Transl Med 2014.

The advantage of such a screening process is in particular the possibility of selecting active agents of interest in the field of skincare and in particular anti-aging.

In addition, the screening process according to the invention makes it possible to identify new active agents for preventing and/or treating the signs of skin aging, and in particular active agents for preventing and/or treating esthetic defects resulting from a loss of connective tissue, for instance a loss of adipose tissue.

The examples and figures that follow are provided as illustrations which do not limit the field of the invention.

### FIGURES

FIG. 1: View of the region from which the fibroblasts are taken, of the dermo-hypodermic junction.

FIG. 2: Demonstration of the differentiation of fibroblasts of the dermo-hypodermic junction into adipocyte cells in a cell model according to example 1.

FIG. 3: Demonstration of the differentiation of fibroblasts of the dermo-hypodermic junction into osteoblast cells in a cell model according to example 1.

FIG. 4: Demonstration of the differentiation of fibroblasts of the dermo-hypodermic junction into chondroblast cells in a spheroid cell model according to example 1.

FIG. 5: Demonstration of the differentiation of fibroblasts of the dermo-hypodermic junction into adipocyte cells in a tissue model according to example 2.

FIG. 6: Demonstration of the differentiation of fibroblasts Fp (outside the invention), Fr (outside the invention), and F-DHJ (according to the invention) into adipocyte cells in a cell model.

FIG. 7: Demonstration of the differentiation of fibroblasts Fp (outside the invention), Fr (outside the invention), and F-DHJ (according to the invention) into osteoblast cells in a cell model.

FIG. 8: Demonstration of the differentiation of fibroblasts Fp (outside the invention), Fr (outside the invention), and F-DHJ (according to the invention) into chondroblast cells in a cell model.

### EXAMPLE 1: PROCESS FOR PREPARING A CELL MODEL OBTAINED FROM DHJ FIBROBLASTS AND VALIDATION OF ITS USE AS A TOOL FOR SCREENING ACTIVE AGENTS THAT PROMOTE THE DIFFERENTIATION OF DHJ FIBROBLASTS INTO ADIPOCYTE, OSTEOBLAST AND CHONDROBLAST CELLS

1) Taking Fibroblasts from the DHJ

The fibroblasts of the dermo-hypodermic junction (F-DHJ) were isolated from non-defatted human skin. These samples are collected after breast reduction for esthetic reasons. The F-DHJs are isolated from the connective trabeculae present at the dermo-hypodermic junction. The latter are taken using tweezers and scissors. (see FIG. 1)

After comminution, the fragments of dermis are digested under the action of type II collagenase at 0.2% (Gibco) at 37° C.

The cells are then amplified in MEM medium—10% fetal calf serum supplemented with glutamine, sodium pyruvate, non-essential amino acids, penicillin, streptomycin and fungizone, under a moist atmosphere, at 37° C. and 5% CO₂.

2) Method for Identifying Fibroblasts of the DHJ (Verification of the Cellular Phenotype by RT-qPCR)

After amplification of the fibroblasts (between 7 and 10 population doublings), the mRNAs are extracted on QlAgen column according to the instructions given by the supplier.

The probes considered to be differentially expressed had to have a fold change of >2 for a p-value<0.05.

The molecular signature of the cells is verified according to the method below. Thus, the F-DHJs will exhibit relative levels of expression of ACAN, Col11a1, FGF9, UCP2 and KLF9 consistent with those summarized in the tables below.

TABLE 1

| | Fp vs FDHJ | |
|---|---|---|
| | (change in number of times) | |
| | RNA | Conclusion |
| ACAN | −5.38 | Upregulated in FDHJs |
| COL11A1 | −24.43 | |
| FGF9 | −3.50 | |
| UCP2 | 4.36 | Downregulated in FDHJs |

| | Fr vs FDHJ | |
|---|---|---|
| | (change in number of times) | |
| | RNA | Conclusion |
| KLF9 | −2.06 | Upregulated in FDHJs |

Fp: papillary fibroblast,
Fr: reticular fibroblast,
FDHJ: fibroblast of the dermo-hypodermic junction

| | UCP2 | COL11A1 | ACAN | FGF9 | KLF9 |
|---|---|---|---|---|---|
| FDHJ | Negative = | Positive = | Positive = | Positive = | Positive = |
| fibroblast | Decreased | Increased | Increased | Increased | Increased |

Table of primers (QIAgen—quantitech primer assay) used for RT-qPCR validations

| Name | Ref |
|---|---|
| ACAN | QT00001365 |
| Col XI | QT00088711 |
| FGF9 | QT00000091 |
| GAPDH | QT01192646 |
| UCP2 | QT00014140 |

3) Process for Preparing a Cell Model According to the Invention

a. 2D Cell Model

The fibroblasts of the dermo-hypodermic junction are seeded in a Petri dish at 1400 cells per cm² in a medium of MEM culture medium—10% fetal calf serum supplemented with glutamine, sodium pyruvate, non-essential amino acids, penicillin, streptomycin and fungizone, under a moist atmosphere, at 37° C. and 5% CO₂. The fibroblasts of the dermo-hypodermic junction are cultured for 48 hours after reaching confluence.

At the end of this process, a cell model according to the invention is thus obtained.

b. Cell Model in Spheroid Form

The fibroblasts of the dermo-hypodermic junction are seeded in a Petri dish at 1400 cells per cm² in a medium of MEM culture medium—10% fetal calf serum supplemented with glutamine, sodium pyruvate, non-essential amino acids, penicillin, streptomycin and fungizone, under a moist atmosphere, at 37° C. and 5% CO₂. The fibroblasts of the dermo-hypodermic junction are cultured for 48 hours after reaching confluence.

48 hours after reaching confluence, spheroids are formed after centrifugation of 100 000 DHJ cells.

At the end of this process, a cell model in spheroid form according to the invention is thus obtained.

4) Validation of the Use of the Cell Model According to the Invention as Tool for Screening Active Agents:

a. That Promote the Differentiation of DHJ Fibroblasts into Adipocyte

In order to validate the cell model produced in paragraph 3) a. for use as a tool for screening active agents that promote differentiation of F-DHJs into adipocytes, a positive control is used that consists of a mixture of active agents comprising indometacin, IBMX and dexamethasone, known for their pro-differentiating activities of fibroblasts into adipocytes (Peiffer et al., Leukemia 2007 April; 21 (4): 714-24).

The culture medium in point 3) a. is therefore substituted by a cocktail composed of: 60 μM indometacin/0.5 mM IBMX/10⁻⁶ M dexamethasone diluted in DMEM/20% fetal calf serum. The cells are kept in culture for 3 weeks in the presence of the differentiation induction cocktail. The media are renewed 3 times a week.

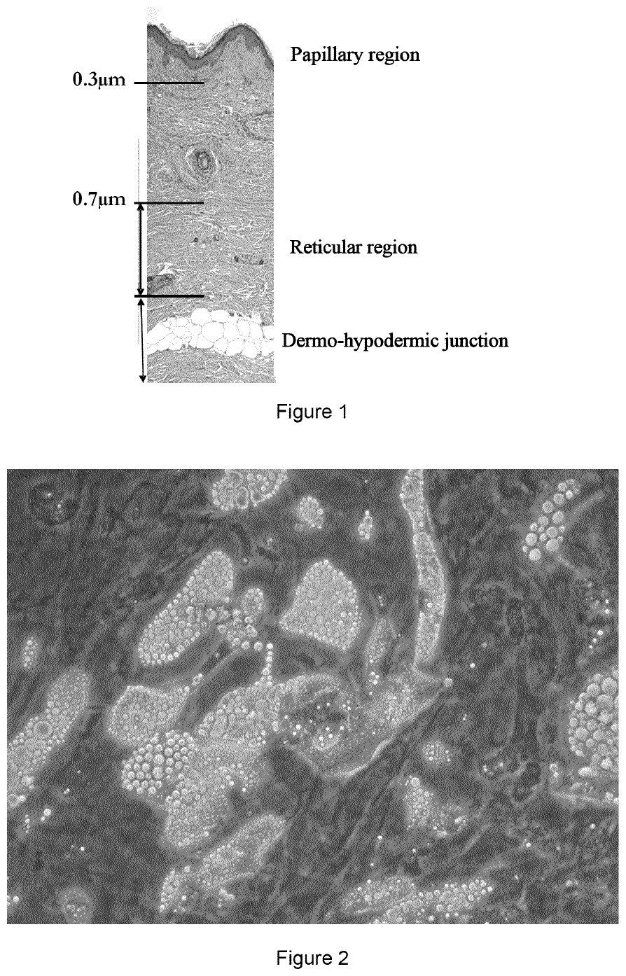

At the end of the 3 weeks of induction, the cell culture is stopped by fixation in paraformaldehyde at 4%. The detection of cells oriented towards an adipocyte differentiation profile is carried out under a microscope. Highly refractive spheres are observable inside the cells. Oil Red O staining, known to stain lipid droplets present inside the adipocytes red, can also be performed. (see FIG. 2)

Conclusion: differentiation of DHJ fibroblasts into adipocytes is observed; thus, this model can be used as a tool for screening active agents that promote the differentiation of DHJ fibroblasts into adipocytes.

b. That Promote the Differentiation of DHJ Fibroblasts into Osteoblasts

In order to validate the cell model produced in paragraph 3) a. for use as a tool for screening active agents that promote differentiation of F-DHJs into osteoblasts, a positive control is used that consists of a mixture of active agents comprising 2β-glycerophosphate, ascorbate 2-phosphate and dexamethasone, known for their pro-differentiating activities of fibroblasts into osteoblasts (Peiffer et al., Leukemia 2007 April; 21 (4): 714-24).

The culture medium in point 3) a. is therefore substituted by a cocktail composed of: 2 mM 2β-glycerophosphate, 0.15 mM ascorbate 2-phosphate, 10⁻⁷M dexamethazone and 10% fetal calf serum. The cells are kept in culture for 3 weeks in the presence of the differentiation induction cocktail. The media are renewed 3 times a week.

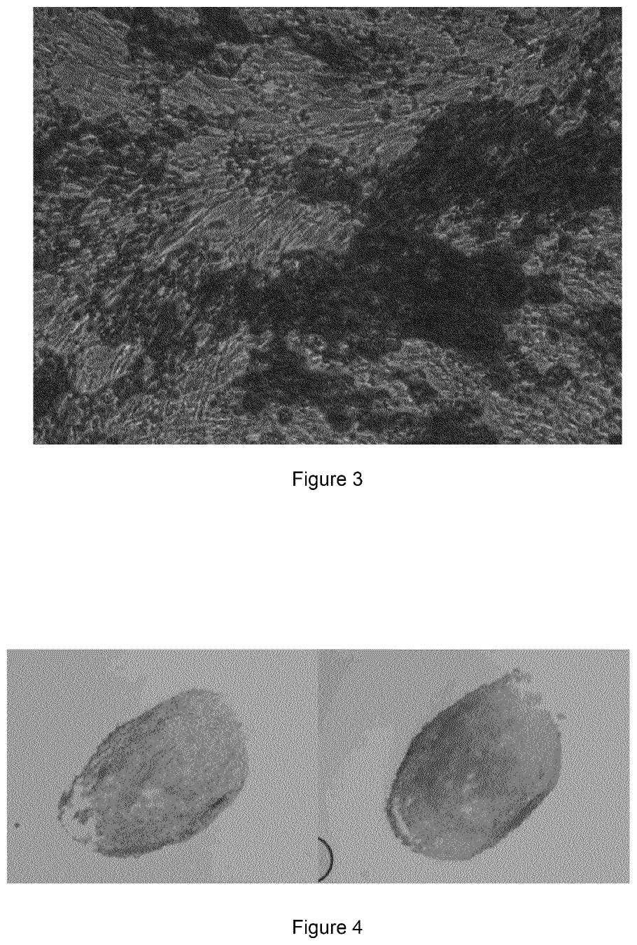

At the end of the 3 weeks of induction, the cell culture is stopped by fixation in paraformaldehyde at 4%. The detection of cells oriented towards an osteoblast differentiation profile is carried out by means of alizarin staining. This product stains calcified extracellular matrices red. (see FIG. 3)

Conclusion: differentiation of DHJ fibroblasts into osteoblasts is observed; thus, this model can be used as a tool for screening active agents that promote the differentiation of DHJ fibroblasts into osteoblasts.

c. That Promote the Differentiation of DHJ Fibroblasts into Chondroblasts

In order to validate the cell model produced in paragraph 3) b. for use as a tool for screening active agents that promote differentiation of F-DHJs into chondroblasts, a positive control is used that consists of a mixture of active agents comprising insulin, transferrin, sodium selenite, linoleic acid, oleic acid, bovine serum albumin, sodium pyruvate, ascorbate 2-phosphate, and dexamethazone, known for their pro-differentiating activities of fibroblasts into chondroblasts (Mesenchymal and hematopoietic stem cells form a unique bone marrow niche, Mendez-Ferrer S, Michurina T V, Ferraro F, Mazloom A R, Macarthur B D, Lira S A, Scadden D T, Ma'ayan A, Enikolopov G N, Frenette P S, Nature. 2010 Aug. 12; 466(7308):829-34. doi: 10.1038/nature09262).

24 hours after forming the spheroids, the culture medium from point 3) b. is thus replaced by an induction cocktail composed of 0.5 μg/ml of insulin, 0.5 μg/ml of transferrin, 0.5 ng/ml of sodium selenite, 6.25 μg/ml of linoleic acid, 6.25 μg/ml of oleic acid, 1.25 mg/ml of bovine serum albumin, 1 mmol/l of sodium pyruvate, 0.17 mmol/l of ascorbate 2-phosphate, 0.1 μmol/l of dexamethazone, 0.35 mmol/l of proline and 0.01 μg/ml of TGF-β. The medium is renewed 3 times a week for 2 weeks.

The cultures are stopped by freezing the spheroids after inclusion in OCT. The spheroids are then cut with a microtome at 5 μm. The differentiation of F-DHJ into chondroblasts is demonstrated by staining with toluidine blue supplemented by staining with safranin O (see FIG. 4).

Conclusion: differentiation of DHJ fibroblasts into chondroblasts is observed; thus, this model can be used as a tool for screening active agents that promote the differentiation of DHJ fibroblasts into chondroblasts.

### EXAMPLE 2: PROCESS FOR PREPARING A TISSUE MODEL ON COLLAGEN SPONGE FROM DHJ FIBROBLASTS AND VALIDATION OF ITS USE AS A TOOL FOR SCREENING ACTIVE AGENTS THAT PROMOTE THE DIFFERENTIATION OF DHJ FIBROBLASTS INTO ADIPOCYTE CELLS

1) Taking Fibroblasts from the DHJ (According to Example 1)

2) Method for Identifying Fibroblasts of the DHJ (Verification of the Cellular Phenotype by RT-qPCR) (According to Example 1)

3) Process for Preparing a Tissue Model According to the Invention

The cells of the dermo-hypodermic junction are seeded at 250 000 cells per sponge (Symatese Biomaterials) in the MEM culture medium—10% fetal calf serum supplemented with glutamine, sodium pyruvate, non-essential amino acids, penicillin, streptomycin and fungizone, in a moist atmosphere, at 37° C. and 5% CO₂. The cultures are maintained for 14 days.

At the end of this process, a tissue model according to the invention is thus obtained.

4) Validation of the Use of the Tissue Model According to the Invention as Tool for Screening Active Agents:

In order to validate the tissue model produced in paragraph 3) for use as a tool for screening active agents that promote differentiation of F-DHJs into adipocytes, a positive control is used that consists of a mixture of active agents comprising indometacin, IBMX and dexamethasone, known for their pro-differentiating activities of fibroblasts into adipocytes (Peiffer et al., Leukemia 2007 April; 21 (4): 714-24).

The culture medium in point 3) is therefore substituted by a cocktail composed of: 60 μM indometacin/0.5 mM IBMX/10⁻⁶ M dexamethasone diluted in DMEM/20% fetal calf serum. The induction of differentiation is maintained for 3 weeks. The media are renewed 3 times a week.

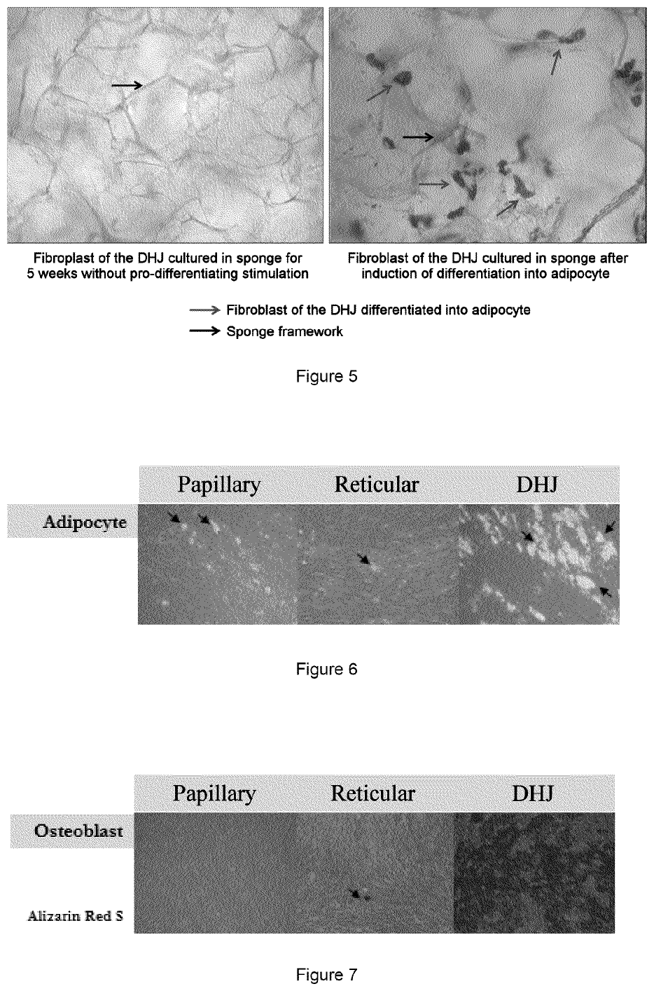

At the end of the 3 weeks of induction, the cell culture is stopped by fixation in paraformaldehyde at 4%. The detection of cells oriented towards an adipocyte differentiation profile is carried out under a microscope after staining with Oil Red 0, known to stain lipid droplets present inside the adipocytes red. (see FIG. 5)

Conclusion: differentiation of DHJ fibroblasts into adipocytes is observed; thus, this model can be used as a tool for screening active agents that promote the differentiation of DHJ fibroblasts into adipocytes.

### EXAMPLE 3: COMPARATIVE, OUTSIDE THE INVENTION: TISSUE MODEL ON COLLAGEN LATTICE OBTAINED FROM DHJ FIBROBLASTS

The lattices were prepared following the previously published protocol (Asselineau et al.—Exp Cell res, 1985). 10⁶ F-DHJ fibroblasts were included in a bovine type I collagen solution (Symatése Biomateriaux).

In order to study the sensitivity of the tissue model on lattice (outside of the invention) in the context of its implementation as a tool for screening active agents that promote the differentiation of DHJ fibroblasts into adipocytes, use is made, as for examples 1), 4) a. and 2), 4), of a positive control consisting of a mixture of indometacin, IBMX and dexamethasone, known for their pro-differentiating activities of fibroblasts into adipocytes, (Peiffer et al., Leukemia 2007 April; 21(4): 714-24).

Thus, after 4 days of organization and contraction of the lattice, the culture medium was substituted with an induction cocktail composed of: 60 μM indometacin/0.5 mM IBMX/10⁻⁶ M dexamethasone diluted in DMEM/20% fetal calf serum. The induction of differentiation is maintained for 3 weeks. The media are renewed 3 times a week.

At the end of the 3 weeks of induction, the cell culture is stopped by freezing the lattices after inclusion in OCT. The lattices are then cut with a microtome at 5 μm. The differentiation of the F-DHJs into adipocytes is demonstrated using Oil Red O staining. In this induction condition, the differentiation of the F-DHJs into adipocytes does not occur. It was not possible to demonstrate a staining of the adipocyte cells.

Conclusion: no differentiation of DHJ fibroblasts into adipocytes is observed; thus, this model is not sufficiently sensitive to be able to be used as a tool for screening active agents that promote the differentiation of DHJ fibroblasts into adipocytes.

### EXAMPLE 4: COMPARATIVE, OUTSIDE THE INVENTION: CELL MODELS OBTAINED FROM PAPILLARY OR RETICULAR FIBROBLASTS

1) Process for Preparing Cell Models Outside the Invention

The process carried out is identical to that described above in example 1, point 3), in which the DHJ fibroblasts have been replaced either by papillary fibroblasts or by reticular fibroblasts.

At the end of this process this thus gives a cell model outside the invention obtained from papillary fibroblasts and a cell model outside the invention obtained from reticular fibroblasts.

2) Study of the Use of Cell Models Outside the Invention as Tool for Screening Active Agents:

a. That Promote the Differentiation of Papillary or Reticular Fibroblasts into Adipocyte

The process carried out is identical to that described in example 1, point 4) a.

The detection of cells oriented towards an adipocyte differentiation profile is carried out under a microscope. Small refractive spheres signifying the presence of lipid vacuoles are observed inside a small number of cells in the cultures established from papillary and reticular fibroblasts (see FIG. 6). Counter-staining with Oil red O validates the fact that these vacuoles are lipid in nature.

Conclusion: a very small amount of differentiation of the papillary or reticular fibroblasts into adipocytes is observed; thus, the cell models obtained from papillary or reticular fibroblasts are not sufficiently sensitive to be able to be used as a tool for screening active agents that promote the differentiation of these fibroblasts into adipocytes.

b. That Promote the Differentiation of Papillary or Reticular Fibroblasts into Osteoblasts

The process carried out is identical to that described in example 1, point 4) b.

The detection of cells oriented towards an osteoblast differentiation profile is carried out by means of alizarin staining. This product stains calcified extracellular matrices red. No red staining is observed in the case of the cell model obtained from papillary fibroblasts, and therefore there is an absence of calcified extracellular matrix. In the case of the cell model obtained from reticular fibroblasts, very small traces of red staining indicating that the extracellular matrix is calcified are observed (see FIG. 7). Thus, the papillary and reticular fibroblasts have a very weak propensity to differentiate into osteoblasts.

Conclusion: a very small amount of differentiation of the papillary or reticular fibroblasts into osteoblasts is observed; thus, the cell models obtained from papillary or reticular fibroblasts are not sufficiently sensitive to be able to be used as a tool for screening active agents that promote the differentiation of these fibroblasts into osteoblasts.

c. That Promote the Differentiation of Papillary or Reticular Fibroblasts into chondroblasts

The process carried out is identical to that described in example 1, point 4) c.

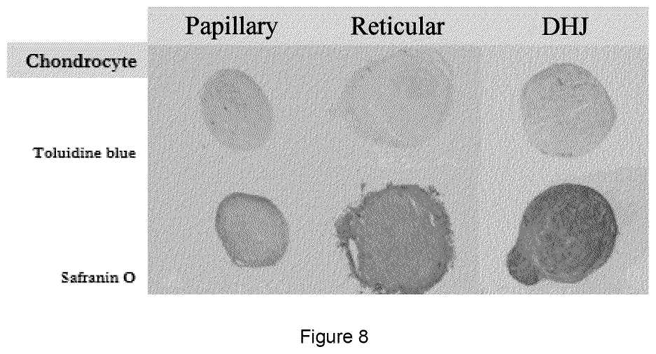

The differentiation of the papillary or reticular fibroblasts into chondroblasts is demonstrated using staining with toluidine blue, supplemented with staining with safranin O. Pale blue and pale red staining is observed in the case of the cell models obtained from papillary and reticular fibroblasts, while dark blue staining is observed at the periphery, and also dark red staining in the case of the cell model obtained from fibroblasts of the DHJ. (see FIG. 8)

Conclusion: a very small amount of differentiation of the papillary or reticular fibroblasts into chondroblasts is observed; thus, the cell models obtained from papillary or reticular fibroblasts are not sufficiently sensitive to be able to be used as a tool for screening active agents that promote the differentiation of these fibroblasts into chondroblasts.

The table below summarizes the differentiation potential of the sub-populations of fibroblasts tested (Fp, Fr or F-DHJ) in adipocyte, osteoblast and chondroblast cells.

TABLE 2

| | Fp | Fr | F-DHJ |
|---|---|---|---|
| Chondrocyte | + | +/− | ++ |
| Adipocyte | + | +/− | +++ |
| Osteoblast | − | +/− | +++ |

1. An in vitro process for preparing a cell model comprising at least one step of culturing fibroblasts of the dermo-hypodermic junction.

2. The process as claimed in claim 1, wherein the fibroblasts are cultured between 24 h and 72 h after reaching at least 80% confluence.

3. The process as claimed in claim 1, also comprising a step of centrifugation of the fibroblasts obtained at the end of said culture step.

4. An in vitro process for preparing a tissue model comprising at least one step of culturing fibroblasts of the dermo-hypodermic junction on collagen sponge.

5. The process as claimed in claim 4, wherein said fibroblasts are cultured for 10 to 20 days.

6. The in vitro process for preparing a cell model as claimed in claim 1 or an in vitro process for preparing a tissue model comprising at least one step of culturing fibroblasts of the dermo-hypodermic junction, also comprising a step of identification of a dermal fibroblast as a fibroblast of the dermo-hyperdermic junction, said identification step being prior to said culture step and comprising:
a) providing a biological sample comprising at least one dermal fibroblast,
b) measuring, in the biological sample provided in step a), the level of an expression product of at least one gene selected from the group consisting of the genes UCP2, ACAN, FGF9 and COL11A1, and the level of an expression product of the gene KLF9,
c) identifying the dermal fibroblast of step a) as a fibroblast of the dermo-hypodermic junction when:
1) (i) the level of the expression product of the gene UCP2 is decreased relative to a control level,
(ii) the level of the expression product of the gene ACAN is increased relative to a control level,
(iii) the level of the expression product of the gene FGF9 is increased relative to a control level, and/or
(iv) the level of the expression product of the gene COL11A1 is increased relative to a control level,
and
2) the level of the expression product of the gene KLF9 is increased relative to a control level,
wherein the control levels of 1(i), 1(ii), 1(iii) and 1(iv) being respectively the level of the expression product of the genes UCP2, ACAN, FGF9 and COL11A1 in a dermal fibroblast known to be a papillary fibroblast and the control level of 2) being the level of the expression product of the gene KLF9 in a dermal fibroblast known to be a reticular fibroblast.

7. An in vitro cell model obtainable according to the process as defined in claim 1.

8. An in vitro tissue model obtainable according to claim 4.

9. The cell model as claimed in claim 7 or tissue model obtainable by an in vitro process comprising at least one step of culturing fibroblasts of the dermo-hypodermic junction on collagen sponge, comprising fibroblasts of the dermo-hypodermic junction for which:
1) (i) the level of the expression product of the gene UCP2 is decreased relative to a control level,
(ii) the level of the expression product of the gene ACAN is increased relative to a control level,
(iii) the level of the expression product of the gene FGF9 is increased relative to a control level, and/or
(iv) the level of the expression product of the gene COL11A1 is increased relative to a control level,
and
2) the level of the expression product of the gene KLF9 is increased relative to a control level,
wherein the control levels of 1(i), 1(ii), 1(iii) and 1(iv) being respectively the level of the expression product of the genes UCP2, ACAN, FGF9 and COL11A1 in a dermal fibroblast known to be a papillary fibroblast and the control level of 2) being the level of the expression product of the gene KLF9 in a dermal fibroblast known to be a reticular fibroblast.

10. The use of in vitro cell or tissue models as defined in claim 7 as tool for screening for active agents that promote the differentiation of fibroblasts of the dermo-hypodermic junction into adipocyte, osteoblast and/or chondroblast cells.

11. A process for screening active agents that promote the differentiation of fibroblasts of the dermo-hypodermic junction into adipocyte, osteoblast and/or chondroblast cells, comprising the following steps:
i. providing a cell as defined in claim 7,
ii. bringing said model into contact with at least one agent to be screened,
iii. carrying out a qualitative and/or quantitative measurement of the expression of at least one marker of the adipocyte, osteoblast and/or chondrocyte cells or of their biological activity (activities), then
iv. comparing the measurement carried out in step iii) with that obtained from a control.

12. A process for screening active agents that promote the differentiation of fibroblasts of the dermo-hypodermic junction into adipocyte, osteoblast and/or chondroblast cells, comprising the following steps:
i. providing a as defined in claim 8,
ii. bringing said model into contact with at least one agent to be screened,
iii. carrying out a qualitative and/or quantitative measurement of the expression of at least one marker of the adipocyte, osteoblast and/or chondrocyte cells or of their biological activity (activities), then
iv. comparing the measurement carried out in step iii) with that obtained from a control.

13. The process as claimed in claim 1, wherein the fibroblasts are cultured for 48 h after reaching at least 80% confluence.

14. The process as claimed in claim 2, also comprising a step of centrifugation of the fibroblasts obtained at the end of said culture step.

15. The in vitro process for preparing a cell model as claimed in claim 2, also comprising a step of identification of a dermal fibroblast as a fibroblast of the dermo-hyperdermic junction, said identification step being prior to said culture step and comprising:
a) providing a biological sample comprising at least one dermal fibroblast,
b) measuring, in the biological sample provided in step a), the level of an expression product of at least one gene selected from the group consisting of the genes UCP2, ACAN, FGF9 and COL11A1, and the level of an expression product of the gene KLF9,
c) identifying the dermal fibroblast of step a) as a fibroblast of the dermo-hypodermic junction when:
1) (i) the level of the expression product of the gene UCP2 is decreased relative to a control level,
(ii) the level of the expression product of the gene ACAN is increased relative to a control level,
(iii) the level of the expression product of the gene FGF9 is increased relative to a control level, and/or
(iv) the level of the expression product of the gene COL11A1 is increased relative to a control level,
and
2) the level of the expression product of the gene KLF9 is increased relative to a control level,
wherein the control levels of 1(i), 1(ii), 1(iii) and 1(iv) being respectively the level of the expression product of the genes UCP2, ACAN, FGF9 and COL11A1 in a dermal fibroblast known to be a papillary fibroblast and the control level of 2) being the level of the expression product of the gene KLF9 in a dermal fibroblast known to be a reticular fibroblast.

16. The in vitro process for preparing a cell model as claimed in claim 3, also comprising a step of identification of a dermal fibroblast as a fibroblast of the dermo-hyperdermic junction, said identification step being prior to said culture step and comprising:
a) providing a biological sample comprising at least one dermal fibroblast,
b) measuring, in the biological sample provided in step a), the level of an expression product of at least one gene selected from the group consisting of the genes UCP2, ACAN, FGF9 and COL11A1, and the level of an expression product of the gene KLF9,
c) identifying the dermal fibroblast of step a) as a fibroblast of the dermo-hypodermic junction when:
1) (i) the level of the expression product of the gene UCP2 is decreased relative to a control level,
(ii) the level of the expression product of the gene ACAN is increased relative to a control level,
(iii) the level of the expression product of the gene FGF9 is increased relative to a control level, and/or
(iv) the level of the expression product of the gene COL11A1 is increased relative to a control level,
and
2) the level of the expression product of the gene KLF9 is increased relative to a control level,
wherein the control levels of 1(i), 1(ii), 1(iii) and 1(iv) being respectively the level of the expression product of the genes UCP2, ACAN, FGF9 and COL11A1 in a dermal fibroblast known to be a papillary fibroblast and the control level of 2) being the level of the expression product of the gene KLF9 in a dermal fibroblast known to be a reticular fibroblast.

17. The in vitro process for preparing a tissue model as claimed in claim 4, also comprising a step of identification of a dermal fibroblast as a fibroblast of the dermo-hyperdermic junction, said identification step being prior to said culture step and comprising:
a) providing a biological sample comprising at least one dermal fibroblast,
b) measuring, in the biological sample provided in step a), the level of an expression product of at least one gene selected from the group consisting of the genes UCP2, ACAN, FGF9 and COL11A1, and the level of an expression product of the gene KLF9,
c) identifying the dermal fibroblast of step a) as a fibroblast of the dermo-hypodermic junction when:
1) (i) the level of the expression product of the gene UCP2 is decreased relative to a control level,
(ii) the level of the expression product of the gene ACAN is increased relative to a control level,
(iii) the level of the expression product of the gene FGF9 is increased relative to a control level, and/or
(iv) the level of the expression product of the gene COL11A1 is increased relative to a control level,
and
2) the level of the expression product of the gene KLF9 is increased relative to a control level,
wherein the control levels of 1(i), 1(ii), 1(iii) and 1(iv) being respectively the level of the expression product of the genes UCP2, ACAN, FGF9 and COL11A1 in a dermal fibroblast known to be a papillary fibroblast and the control level of 2) being the level of the expression product of the gene KLF9 in a dermal fibroblast known to be a reticular fibroblast.

18. The in vitro process for preparing a tissue model as claimed in claim 5, also comprising a step of identification of a dermal fibroblast as a fibroblast of the dermo-hyperdermic junction, said identification step being prior to said culture step and comprising:
a) providing a biological sample comprising at least one dermal fibroblast,
b) measuring, in the biological sample provided in step a), the level of an expression product of at least one gene selected from the group consisting of the genes UCP2, ACAN, FGF9 and COL11A1, and the level of an expression product of the gene KLF9,
c) identifying the dermal fibroblast of step a) as a fibroblast of the dermo-hypodermic junction when:
1) (i) the level of the expression product of the gene UCP2 is decreased relative to a control level,
(ii) the level of the expression product of the gene ACAN is increased relative to a control level,
(iii) the level of the expression product of the gene FGF9 is increased relative to a control level, and/or
(iv) the level of the expression product of the gene COL11A1 is increased relative to a control level,
and
2) the level of the expression product of the gene KLF9 is increased relative to a control level,
wherein the control levels of 1(i), 1(ii), 1(iii) and 1(iv) being respectively the level of the expression product of the genes UCP2, ACAN, FGF9 and COL11A1 in a dermal fibroblast known to be a papillary fibroblast and the control level of 2) being the level of the expression product of the gene KLF9 in a dermal fibroblast known to be a reticular fibroblast.

19. An in vitro cell model obtainable according to the process as defined in claim 2.

20. An in vitro tissue model obtainable according to claim 5.
